(19) United States
(12) Patent Application Publication
(10) Pub. No.: US 2011/0008301 A1
(43) Pub. Date: Jan. 13, 2011

(54) HUMAN ADIPOSE DERIVED INSULIN MAKING MESENCHYMAL STEM CELLS FOR TREATING DIABETES MELLITUS

(76) Inventors: H. L. Trivedi, Ahmedabad (IN); Aruna Vanikar, Ahmedabad (IN)

Correspondence Address: Davidson, Davidson & Kappel, LLC, 485 7th Avenue, 14th Floor, New York, NY, 10018, US

(21) Appl. No.: 12/922,624

(22) PCT Filed: Mar. 13, 2009
(86) PCT No.: PCT/IN09/00174

§ 371 Date: Sep. 14, 2010

(30) Foreign Application Priority Data
Mar. 15, 2008 (IN) 211/MUM/2008

(51) Int. Cl. A61K 35/12; C12N 5/0775; C12P 21/00; A61P 3/10; A61P 17/02

(52) U.S. Cl. 424/93.7; 435/377; 435/70.1

(57) ABSTRACT

The invention provides a novel therapeutic composition comprising of insulin producing mesenchymal stem cells obtained from human adipose tissue along with Hematopoietic stem cells for the treatment of diabetic patients especially insulinopenic patients. The invention also describes a simple and efficient process for the isolation, proliferation and differentiation of insulin producing mesenchymall stem cells from human adipose tissue. Unfiltered extract of adipose tissue is used in the process with a medium totally free from xenogenic material; the serial passages of the cells are avoided in the process.

### FIELD OF INVENTION

The present invention provides a composition comprising of insulin making mesenchymal stem cells obtained from human adipose tissue for the treatment of diabetes. The invention also describes a simple process of obtaining the insulin making mesenchymal stem cells from human adipose tissue.

### BACKGROUND OF THE INVENTION

Diabetes mellitus is a chronic metabolic disorder characterized by high blood sugar (ie. glucose) levels. This is either caused by shortage of insulin, a hormone that regulates blood sugar levels or by the body's failure to respond to the insulin secreted by the beta cells of the pancreas.

Diabetes is considered a major health hazard and the incidence of diabetes is increasing rapidly both in the developed and the developing countries; almost a diabetes epidemic is underway. According to WHO estimate the number of people with diabetes world wide in 2000 was 171 million which is expected to increase to at least 366 million by the year 2030. India is predicted to be ‘diabetic capital’ with maximum number of people suffering from diabetes.

Diabetes has become one of the major causes of premature illness and death in most countries, a diabetic runs the extra risk of getting cardiac and kidney failure problems. At present therapeutic options available to these patients are life long-medications in the form of oral hypoglycemic agents and recombinant insulin preparations. In spite of taking these medications and stringent dietary and life style control, 30% develop kidney failure and/or cardiac problems along with minor disorders like neuropathy, retinopathy, foot ulcer etc.

Diabetes also has an adverse impact on economy. According to WHO estimates, 25% of family income of an average Indian family with an adult diabetes is spent on diabetic care.

The two basic forms of diabetes mellitus are
i) Type 1 or insulin dependent:—Patients with this type of diabetes produce very little or no insulin and require daily injections of insulin to control blood sugar levels. Yet these patients have very unstable blood sugar levels causing complications.
ii) Type 2 or non-insulin dependent diabetes mellitus:—Patients with this type of diabetes cannot use insulin effectively and often require oral drugs and less frequently insulin in order to achieve good metabolic control.

Diabetes has been treated with a number of drugs which include Insulins, Sulfonyl ureas (Chlorpropamide, Glipizide) Meglitinides (Repaglinide) Alpha Glucosidase inhibitors (Acarbose), Biguanides (Metformin) and Thiazolidinediones (Pioglitazone). These drugs either enhance insulin secretion by stimulating pancreatic beta cells or delay absorption of glucose from gastrointestinal tract or target ‘insulin resistance’ which is of critical importance in type 2 diabetes.

In spite of the availability of a number of anti-diabetic drugs, the control and prevention of complications of diabetes remains far from satisfactory.

This could be due to the adverse effects of the drugs like unexpected hypoglycemic episodes, ineffective sugar control or poor patient compliance, especially with injectable insulin therapy.

Recent advances in medical science has shown that ‘stem cells’ have the potential to radically change the treatment of human diseases. They could revolutionize the therapy of multiple medical conditions which include malignancies, spinal cord injuries and in regenerative medicine, neurological disorders, autoimmune disorders like, systemic lupus erythematosus, skin disorders like psoariasis, etc, cardiac disorders like acute/chronic heart failure, and even diabetes.

A stem cell is defined as a cell capable of renewing itself, as well as to produce progeny destined to differentiation. Replacement of dying/dead cells of different tissues of the body and blood is regulated by these cells through natural mechanisms.

### Sources of Stem Cells

Embryo i.e. blastocysts
Umbilical cord blood (UCB)
Bone marrow (BM)
Peripheral blood

In addition to these, every tissue has specialized kind of stem cells. Stem cells can now be grown and transformed in to specialized cells consistent with cells of various tissues through cell culture.

Stem cells from the bone marrow are the most studied type of adult stem cells. The two major types of stem cells found in the bone marrow are the ‘hematopoietic stem cells’ which form blood, and ‘stromal’ (mesenchymal) stem cells (MSC) which typically form bone, cartilage, muscle and fat.

Recent research has shown that another possible, yet untapped source of stem cells could be ‘Adipose tissue’. Preliminary studies have shown that stem cells isolated from adipose tissue can differentiate and give rise to many cell types. There is also evidence to show that adult pluripotent stem cells exist in adipose tissue which may have high degree of ‘plasticity’. Adipose tissue derived MSC are similar to bone marrow and cord blood derived MSC with respect to all culture characteristics and functions. They are easily available in large quantum and can be effectively cultured as an alternative source to bone marrow derived MSC. Unfortunately till date, studies of stem cells isolated from adipose tissues are largely restricted to laboratory and are still experimental. There are only sporadic reports of the use of human adipose tissue as a source of stem cells in clinical setting.

The recent success of Edmonton protocol for pancreatic islet transplantation in cases of diabetes has sparked interest in transplantation of insulin producing cells but the main difficulty is obtaining enough donor islet tissue. Therefore multiple approaches are being explored to generate insulin producing cells in vitro. Embryonic stem cells have been promising in this respect, but the potential use of Embryonic stem cells for the treatment of diseases in human is beclouded in controversy because of the ethical issues.(Am. J. Physiol Endocrinol Metab 284,E259-E266,2003). Researchers at Burham Institute of Medical research and the Rebecca and John Moores Cancer Center at the University of California recently demonstrated that adult stem cells in the pancreas can be transformed into insulin producing cells. This was the first proof that beta cell regeneration therapies can be developed for the treatment of diabetes. Researchers at the University of Texas reported that umbilical cord blood derived stem cells can be engineered to synthesize insulin.(Med J. of Cell Proliferation , June, 2007). Kojima et al reported the detection of immunoreactive proinsulin and insuli-positive cells were detected in the liver, adipose tissue and bone marrow of diabetic rats indicating that extrahepatic extrathymic insulin production occurs in more than one species and these observations have implications for strategies for the generation of insulin producing cells for the treatment of diabetes. (Proc Natl Acad. Sci (USA) 2004, 101, 2458-2463). Karnieli et al (Stem Cells, V25, 11, 2837-2844, 2007) have reported generation of insulin-producing cells from human bone marrow mesenchymal stem cells by genetic manipulation.

A method was described for producing an insulin secreting pre-adipocyte stem cell and the cells derived there from by obtaining fat stem cells from a patient; culturing the fat stem cells and suspending them in media together with an insulin gene, and subjecting the fat stem cells to transfection via electroporation (PATENT WO 200708163). However there is no unequivocally accepted protocol for isolation and differentiation of stem cells into insulin producing cells and there is little agreement on the cell type to be studied; the results are inconsistent.

Timper et al isolated human adipose tissue derived mesenchymal cells from healthy donors. During the proliferation period, the cells expressed the stem cell markers nestin, ABCG2, SCF, THY-1, as well as the pancreatic endocrine transcription factor Isl-1. The cells were induced to differentiate into a pancreatic endocrine phenotype by defined culture conditions within three days. Using Quantitative PCR a down regulation of ABCG2 and up regulation of pancreatic developmental transcription factors Isl-1, Ipf-1 and Ngn3 were observed together with induction of the islet hormones insulin, glucagon, and somatostatin. (Biochemical and Biophysical Research Communications 341, 2006, 1135-1140).

It is apparent that the method for isolation and differentiation of insulin producing mesenchymal stem cells from human adipose tissue and administration of the same to a diabetic patient is far from standardized and the results are not consistent. The methods attempted were complicated, cumbersome and costly requiring sophisticated instruments and difficult to carry out in a normal clinical set up. Another problem with the reported techniques is the use of fetal bovine serum (FBS) in the medium—which is a highly variable undefined component. It has the potential of causing adverse reactions in the patients. In fact, it has been reported that many patients who received mesenchymal stromal cells (MSC) as cellular therapy developed anti-fetal calf serum antibodies where MSC culture medium had Fetal Calf Serum as a component. (Sundin et.al , Haematologica, V 92,9 ,1208-1215,2007) Hence there is a definite need for a simple and efficient process for obtaining insulin producing mesenchymal stem cells from human adipose tissue and a proper therapeutic composition based on the adipose derived stem cells for the treatment of diabetic patients particularly insulinopenic diabeteic patients.

### OBJECTIVES AND SUMMARY OF THE INVENTION

The main objective of the present invention is to provide a simple and efficient process for isolating insulin producing mesenchymal stem cells from human adipose tissue and a therapeutic composition based on these stem cells for the treatment of diabetes. Another objective of the invention is to provide a method of isolating insulin producing mesenchymal stem cells from human adipose tissue without using any xenogenic material in the proliferation or differentiation medium. Yet another objective of the invention is to provide a cost-effective technology for isolating insulin producing mesenchymal stem cells from human adipose tissue.

The present invention uses unfiltered but centrifuged extract of adipose tissue for culturing in a proliferation medium free from any xenogenic material but with growth factors and appropriate antibiotics and antifungal agent. Serial passage of the cells avoided and a suitable medium was used for differentiation. After testing for sterility, viability and counts the isolated and cultured insulin making cells optionally with hematopoietic cells are infused preferably in to portal circulation of an insulinopenic diabetic patient.

### DETAILED DESCRIPTION OF THE INVENTION

### 1. Generation Of Insulin Making Mesenchymal Stem Cells From Human Adipose Tissue Derived Mesenchymal Stem Cells

Isolation of MSC after collection from adipose tissue:

After their informed consent is obtained, 2-3 gram of adipose tissue is resected from anterior abdominal wall of donors under local anesthesia after making a small incision on left lateral side below umbilicus. Sutures are taken after hemostasis is secured.

This adipose tissue is collected in proliferation medium with following composition:
1. Dulbecco's modified eagle's medium (DMEM, Sigma, USA) (with high glucose), 1450 mg/L-20 ml
2. 20% human albumin (Reliance Life Sciences, India)-5 ml
3. Fibroblast growth factor-5 ng/Ml-nutrition
4. 1% Sodium pyruvate-buffer
5. Penicillin (200000 units/ml)-20 pi
6. Streptomycin (200000 units/ml)-20 μl
7. Cefotaxime (1 gm/ 5 ml)-10 μl
8. Fluconazole, 100 mg/dl-10 μl

The adipose tissue is minced with knife into tiny pieces. Subsequently collagenase type I, 10 mg/10 ml is added to the above mentioned medium to which the minced tissue is transferred. The entire contents of the medium are processed in Petri dish and after mincing they are placed in incubator at 37° C. with shaker arranged with 100 RPM for 1 hour. Then the entire contents are transferred to 15 ml centrifuge tubes and centrifuged at 780 RPM for 8 minutes. The supernatant and pellets are separately cultured in the proliferation medium on 100 sq. cm and 25 sq. cm. cell+plates (Sarsted, USA), respectively at 37° C. with 5% CO2 for 10 days. Medium is changed every other day.

Then the cells are harvested by means of trypsinization (0.25% trypsin EDTA solution, made up of 0.25% trypsin and 0.2% sodium EDTA powder, Hi Media, India) after washing with 1 N phosphate buffered saline. Collected cells are checked for viability, sterility and cell counts, flow cytometric analysis of cells are carried out. CD 45(Per CP) negative and CD90 (PE)/CD73 (PE) positive tests are carried out. They are also stained by hematoxylin and eosin and further subjected to differentiation in to insulin expressing cells using differentiation medium with following composition.
1. DMEM (glucose-17.5 mM), 25 ml
2. DMEM: F 12: 1:1, 25 ml.
2. Nicotinamide-10 Mm-nutrition
1. Activin A: 2 nM-upregulation of gene expression
2. Exendin 4: 10 nM-upregulation of gene expression
3. Pentagastrin: 10 nM-upregulation of gene expression
4. Hepatocyte Growth Factor (HGF): 100 picoM
5. B-27, N-2, serum supplement: 10 μl each-to prevent proliferation
6. Penicillin (200000 units/ml)-20 μl
7. Streptomycin (200000 units/ml)-20 μl
8. Cefotaxime (1 gm/5 ml)-10 μl
9. Fluconazole, 100 mg/dl-10 μl

The cells are kept in this medium for 3 days and then subjected to isolation by the following technique. 15 ml centrifuge tubes (Sarsted) are taken and 3 ml Ficoll Hypaqe is filled in them. The entire medium along with floating cells is pipetted and layered on Ficoll Hypaqe. They are centrifuged at 800 RPM for 15 minutes. The white ring of cells at the interphase is aspirated and washed with 1 N PBS (Hi Media, India). Cell button is then diluted with equal amount of differentiation medium and subsequently subjected to following measurements after testing for sterility and viability:
1. Pax-6, the key regulator for normal islet cell development by immunofluorescence
2. Isl-1, the gene which upregulates expression of insulin, by immunofluorescence
3. Ipf-1/pdx-1is not only a regulator of (β-cell specific gene expression and function but also is important in self-renewal of β progenitor cells, by immunofluorescence
4. C-peptide assay-by chemiluminescence.

For immunocytochemistry, a technique to identify and study the morphology of these cells, cells transferred on glass slides coated with poly-L-lysine are incubated overnight in DMEM/F12 medium containing 20% human albumin instead of fetal bovine serum (FBS) which is xenogeneic. Non-specific binding is also prevented by incubation with 20% human albumin and not FBS. Cells are incubated with primary antibodies-pax 6, isl-1, ipf-1 which identify them as insulin-making cells. Polyclonal rabbit IgG (used externally for studying the cells made in lab) is used as a secondary antibody instead of Alexa fluor-488 or rhodamine red-coupled straptavidine dye used by others as secondary antibody. These techniques are used only for staining and studying the cells made in lab and are not used anywhere in culturing the cells After testing for sterility, viability, and counts, the insulin making mesenchymal stem cells isolated and cultured from human adipose tissue described as above, are infused preferably , into the portal circulation of an insulinopenic diabetic.

1. A method of isolating and differentiating insulin producing Mesenchymal Stem Cells from Human Adipose tissue, the method comprising steps of:
(a) mincing the adipose tissue into pieces,
(b) adding collagenase type 1 to the pieces,
(c) keeping the contents obtained in step (b) in a shaker placed in an incubator,
(d) centrifuging the contents obtained in step (c) to obtain a supernatant and a pellet,
(e) culturing separately the supernatant and the pellet in culture dishes containing “xenogenic material-free” proliferation medium for about 10 days in CO2 atmosphere at 37° C., the medium comprising DMEM(Dulbecco's modified eagles medium) with high glucose, human albumin, Fibroblast growth factor (FGF),sodium pyruvate buffer, antibiotic, and an antifungal,
(f) replenishing the media without resorting to serial passage,
(g) harvesting the cultured cells by means of trypsinization,
(h) subjecting the harvested cells to at least one test selected from the group consisting of viability, sterility, cell counts, flow cytometric analysis for CD45 negative/90 positive/73 positive events,
(i) subjecting the cells after testing to differentiation of insulin expressing cells using differentiation Medium comprising DMEM-high glucose, DMEM F12,nicotinamide, Activin A, Exendin 4, Pentagastrin, HGF, B27 , N2 (serum supplements), an antibiotics, and an antifungal,
(j) isolating the differentiated cells after at least 3 days, and
(k) testing the isolated cells for sterility and viability, (l) measuring Pax-6, Isl-1, Ipf-1/pdx-I, C-peptide and insulin levels produced by the isolated cells.

2. The method of claim 1, wherein the concentration of human albumin in the proliferation medium is 20%.

3. The method of claim 1, wherein the concentration of the Fibroblast growth factor in the proliferation medium is 5 nanograms/ml.

4. The method of claim 1, wherein the antibiotic of the proliferation medium comprises a combination of penicillin, streptomycin and cefotaxime.

5. The method of claim 1, wherein the antifungal of the proliferation medium is fluconazole.

6. The method of claim 1, wherein the isolation of cells is done by filling centrifuge tube with Ficoll Hypaque, layering the differentiation medium and floating cells on the Ficoll hypaque, centrifuging and aspirating the white ring of cells at an interphase and washing the aspirated white ring of cells with a buffer.

7. A method of treating diabetes comprising administering to a diabetic patient insulin producing mesenchymal stem cells produced and differentiated by the method of claim 1.

8. The method as claimed in claim 7, wherein the administered cells were produced and differentiated by the method of claim 2.

9. The method of claim 7, wherein the administered cells were produced and differentiated by the method of claim 3.

10. The method as claimed in claim 7, wherein the administered cells were produced and differentiated by the method of claim 4.

11. (canceled)

12. The method as claimed in claim 7, wherein the mesenchymal stem cells are administered along with hematopoietic stem cells to the patient.

13. The method as claimed in claims 7, wherein the mesenchymal stem cells along with hematopoietic stem cells are infused preferably into the portal circulation of the diabetic patient for the treatment of diabetes.

14. A therapeutic composition for the treatment of diabetes comprising of human adipose tissue derived insulin making mesenchymal stem cells.

15. The therapeutic composition of claim 15, comprising the human adipose tissue derived insulin making mesenchymal stem cells and haematopoietic stem cells.

16. The therapeutic composition of claim 14, wherein the adipose tissue derived insulin making mesenchymal stem cells are obtained by a process according to claim 1.

17. The method of claim 12, wherein the mesenchymal stem cells and the hematopoietic stem cells are infused preferably into the portal circulation of the patient.

18-21. (canceled)

22. A method of producing insulin in vitro comprising producing insulin by mesenchymal stem cells obtained by the method of claim 1.

23-26. (canceled)

27. A pharmaceutical composition according to claim 14, wherein the mesenchymal stem cells are obtained by a process comprising culturing unfiltered, centrifuged adipose tissue extract comprising a centrifuged pellet and a supernatant, in a xenogenic material free proliferation medium, and periodical replenishment of the medium without the requirement of serial passage or gelatin coated culture plates and with a suitable xenogenic material free differentiation medium.

28. The pharmaceutical composition of claim 27, further comprising hematopoietic stem cells for the treatment of diabetic patients.

29. (canceled)

30. The pharmaceutical composition of claim 27 for the treatment of diabetic wounds.
